(19) United States
(12) Patent Application Publication
(10) Pub. No.: US 2025/0223613 A1
(43) Pub. Date: Jul. 10, 2025

(54) METHOD FOR MAINTAINING AND/OR ENHANCING EFFECT OF ACTIVATOR, METHOD FOR PRODUCING OBJECT CELL, AND DELIVERY CARRIER SET AND KIT

(71) Applicant: KABUSHIKI KAISHA TOSHIBA, Tokyo (JP)

(72) Inventors: Mitsuko ISHIHARA, Setagaya (JP); Emi NOZAKI, Shinagawa (JP); Saeko SARUWATARI, Kawasaki (JP)

(73) Assignee: KABUSHIKI KAISHA TOSHIBA, Tokyo (JP)

(21) Appl. No.: 18/999,936

(22) Filed: Dec. 23, 2024

(30) Foreign Application Priority Data
Jan. 5, 2024 (JP) 2024-000717

(51) Int. Cl. C12N 15/88

(52) U.S. Cl. CPC C12N 15/88

(57) ABSTRACT

According to one embodiment, a method is for maintaining and/or enhances the effect of an activator in a target cell. The method includes bringing a target cell in a first state into contact with a first lipid nanoparticle encapsulating a first activator, generating a target cell in a second state from the target cell in the first state, bringing a second lipid nanoparticle encapsulating a second activator into contact with the target cell in the second state, generating a target cell in a third state from the target cell in the second state, and maintaining and/or enhancing the effect of the activator in the target cell. Each of the first and second lipid nanoparticles is designed to exhibit an affinity appropriate for the corresponding target cell.

### CROSS-REFERENCE TO RELATED APPLICATIONS

This application is based upon and claims the benefit of priority from Japanese Patent Application No. 2024-000717, filed Jan. 5, 2024, the entire contents of which are incorporated herein by reference.

### REFERENCE TO A SEQUENCE LISTING

In accordance with 37 CFR § 1.831-1835 and 37 CFR § 1.77(b) (5), the specification makes reference to a Sequence Listing submitted electronically as a .xml file named “556239US_ST26.xml. The .xml file was generated on Dec. 23, 2024 and is 2,078 bytes in size. The entire contents of the Sequence Listing are hereby incorporated by reference.

### FIELD

Embodiments described herein relate generally to a method for maintaining and/or enhancing the effect of an activator, a method for producing an object cell, and a delivery carrier set and a kit thereof.

### BACKGROUND

The delivery of genes into target cells is widely used in a variety of fields, from basic research to clinical practice, for example, for the treatment and prevention of various diseases, including cancer, as well as for diagnosis, and for the silencing of specific genes and cell reprogramming. Such a delivery of genes can achieve the desired effect by causing the genes delivered into the target cells to exert specific activity in the target cells.

### BRIEF DESCRIPTION OF THE DRAWINGS

FIG. 1 is a cross-sectional view showing an example of a lipid nanoparticle for use in a method according to the first embodiment.

FIG. 2 is a schematic diagram showing the method of the first embodiment.

FIG. 3 is a cross-sectional view showing another example of the lipid nanoparticle for use in the method according to the first embodiment.

FIG. 4 is an image diagram showing a concept of the method of the first embodiment.

FIG. 5 is an image diagram showing a concept of the method of the first embodiment.

FIG. 6 is a schematic diagram showing a method according to the second embodiment.

FIG. 7 is a cross-sectional view showing an example of the lipid nanoparticle for use in the method according to the first embodiment.

FIG. 8 is a cross-sectional view showing an example of the lipid nanoparticle for use in the method according to the first embodiment.

FIG. 9 is a graph showing experimental results.

FIG. 10 is a graph showing experimental results.

FIG. 11 is a graph showing experimental results.

### DETAILED DESCRIPTION

In general, according to one embodiment, a method is for maintaining and/or enhancing the effect of an active agent in a target cell. The method comprises bringing a first lipid nanoparticle encapsulating a first activator into contact with a target cell in a first state, generating a target cell in a second state from the target cell in the first state, bringing a second lipid nanoparticle encapsulating a second activator into contact with the target cell in the second state, generating a target cell in a third state from the target cell in the second state, and maintaining and/or enhancing the effect of the activator in the target cell. The first and second lipid nanoparticles each have a lipid composition designed to exhibit an affinity appropriate for the corresponding target cell in the respective one of the first and second states. The lipid composition of the second lipid nanoparticle is different from the lipid composition of the first lipid nanoparticle.

Embodiments will now be described with reference to the accompanying drawings. In these embodiments, substantially identical structural elements are denoted by the same symbols, and some of the descriptions may be omitted. Note that the drawings are schematic, and the relationship between the thickness and planar dimensions of each part, the ratio of the thickness of each part, etc., may differ from the actual ones.

### First Embodiment

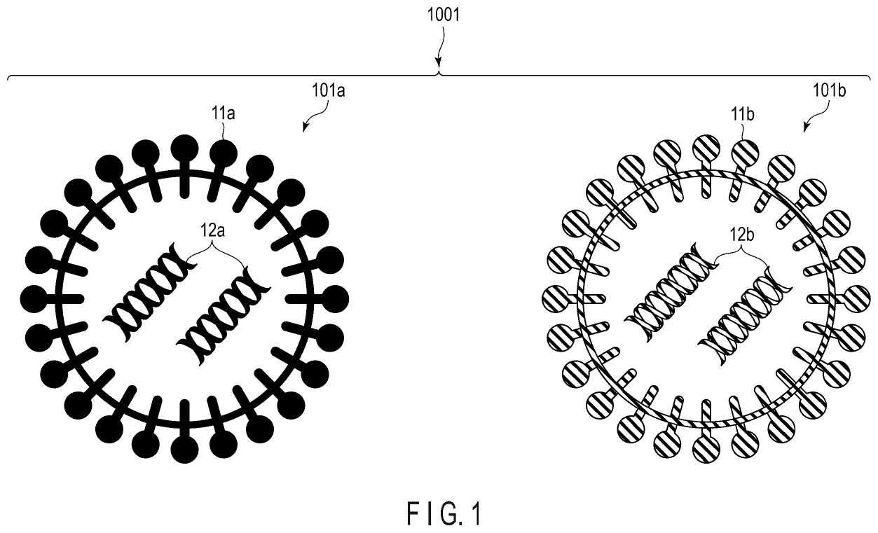

The first embodiment is directed to a method for maintaining and/or enhancing the effect of an activator in target cells. In this method, the target cells are activated using a delivery carrier set 1001 shown in FIG. 1. With reference to FIG. 1, an example of the delivery carrier set will be explained. The delivery carrier set 1001 comprises a first delivery carrier 101a and a second delivery carrier 101b. The first delivery carrier 101a comprises lipid nanoparticles 11a and activators 12a. The lipid nanoparticles 11a are separated by a lipid membrane, and they are liposomes or LNP, for example. The activators 12a are encapsulated inside the lipid nanoparticles 11a. The second delivery carrier 101b comprises lipid nanoparticles 11b and activators 12b. The lipid nanoparticles 11b are separated by lipid membrane, and they are liposomes or LNP, for example. The activators 12b are encapsulated inside the lipid nanoparticles 11b. In the example shown in FIG. 1, the activators 12a and the activators 12b are of types different from each other.

The lipid nanoparticles 11a have an appropriate affinity for the target cell in a first state. Here, the term “appropriate affinity” means, for example, that the affinity is high with the target cell in the first state, as compared to similar delivery carriers of general design under normal and/or general contact conditions. Such appropriate affinity can be achieved by adjusting the lipid composition of the lipid nanoparticle, as will be described in more detail later.

Here, the term “target cell” refers to a cell to which the activator is to be applied. For example, a target cell in the first state can be a target cell in the second state or an object cell by the action of the activator. For example, the target cell in the first state is a cell that is to be changed by the delivery or action of the activator. Such a target cell may be, for example, a diseased cell to be treated by the delivery of the activator, or a material cell that is the source or origin of the object cell to be obtained. The target cell may be selected according to the purpose.

The “object cell” can be, for example, a cell in a state after the target cell as a material cell is brought into contact with an activator, a cell in a state after the activator has acted thereon, a cell in a state after it has been reacted with the activator, or a cell in a state after the activator has been delivered thereto. The type of target cell to be created and/or obtained can be decided by the practitioner as desired. In some cases, such as when the method or steps contained in the method are repeatedly carried out, the object cell can become a further material cell to which an activator should subsequently be delivered or acted thereupon. The term “material cell” can be used interchangeably with the terms “initial material cell”, “material cell in the first state”, and “seed cell”. For example, if multiple activators are used and multiple rounds of delivery are carried out in succession, the material cell is in the first state before delivery, and the material cells, or target cells, can continue to change their properties or characteristics according to the number of repetitions of the processing steps required to obtain the object cell in the end. Here, the word “state” is used for convenience to refer to the state or properties of the material cells, or whether or not it has been brought into contact with a specific activator, and it is also described as a “material cell in the second state”, “material cell in the third state”, . . . , “material cell in the n-th state” or the like, depending on the number of times the activator has been delivered or acted upon (n is an integer greater than or equal to 3). Whether the “target cell” as a “material cell” that has been brought into contact with a specific activator is to be used as a “material cell” for subsequent processing steps or as a “target cell” to be obtained in the end can be determined according to the wishes of the practitioner or according to the subsequent procedures.

As described above, the “object cell” can be a “cell in a state” of the object or a cell to be produced. Examples of object cells can be any desired cells that are composed to have properties that differ from those of the target cell, which is the initial material cell, by being delivered with an activator. Such cells can be any cells formed by being delivered with a gene, for example. Examples of the object cell can be cells used in the laboratory level, basic research fields, therapeutic fields, regenerative medicine fields and the like, such as treated cells, healthy cells, cells treated for disorders or defects, cells in which disorders or defects have been eliminated, modified cells, or reprogrammed cells, artificial pluripotent stem cells such as iPS cells, and the like.

Here, the “activator” may be an active ingredient that has an effect of transforming it into an object cell having different properties, physical properties and/or morphology from the original target cell, or transforming it into a next state, or into an object cell or bringing it into an object state or the like, as it is brought into contact with, delivered and/or acted on the target cell and/or made to react with the target cell. Examples of the activator include substances for recombining the genome of a desired cell, which are substances generally referred to as genes. Specifically, for example, such activators may be natural products, compounds, extracts, nucleic acids, peptides, proteins and the like. For example, the nucleic acids may be nucleic acid fragments, nucleic acid constructs, DNA, RNA and the like. The activator applied to the material cell may be one type or a combination of two or more types. The activity of the first activator and/or the second activator may be, respectively, protein synthesis activity, protein cleavage activity, activity that affects the protein expression state, enzyme activity, anti-cancer activity, reprogramming activity, nucleic acid cleavage activity, gene recombinant activity, and genome editing activity. For example, the first activator and/or second activator may be a nucleic acid substance encoding any gene having an activity selected from the group consisting of protein synthesis activity, protein cleavage activity, anti-cancer activity, reprogramming activity, nucleic acid cleavage activity, gene recombination activity, and genome editing activity. Further, for example, for uniform delivery, it is also preferable that the activator contained in a single liposome should be of a single type. Note here that the activator may be consisted from a single component or multiple components of different types. In the method, multiple types of activators may be used in a single process, or multiple types of activators may be used in multiple processes, respectively. Further, a single type of activator may be used in a single process, or the same type of activator may be used in each of multiple processes.

For example, the activator can be a substance that acts on and/or reacts with the target cell at least at one point or time during or after delivery into the target cell, such as during or after approaching the target cell, contacting the target cell, entering the interior of the target cell, contacting the nucleus of the target cell, or entering the nucleus. The “delivery” of an activator into a cell is, in a narrow sense, to bring the activator to within a distance position where it can act on and/or react with the cell. In a broader sense, this also comprehensively includes the activator coming into contact with the cell, the activator being brought into the cell, the activator being brought into a state where it can act on the cell nucleus, the activator being brought into a state where it can come into contact with the cell, the activator being brought into the cell nucleus, and the like. Further, the “activity” thereof may be the action of the activator on the cell, the reaction between the activator and the cell, or both. For example, the delivery of the activator into the cell may be carried out by introducing the activator into a solution containing the cell. For example, the delivery of the activator into the cell, for example, contacting with the cell, may be carried out by incubation at a certain temperature. The incubation may be carried out while being sent through a flow channel, or it may be carried out while being held in a specific position by a cell trapping mechanism or device, or it may be carried out in a single container. For example, the incubation temperature may be about 35° C. to about 38° C., depending on the type of cell or activator. When the activator is delivered, a specific state of the target cell becomes a state different from the initial state, and/or the target cell becomes an object cell, according to the type and/or condition of the activator and/or target cell.

As described above, the lipid nanoparticle can be a lipid liposome. It can be a particle of a lipid membrane that encapsulates a core of aqueous solution, for example, a particle of a lipid bilayer membrane. For the liposome, any liposome itself known in the conventional art may be utilized. Lipid nanoparticle can be LNP. It can be a particle separated by lipid membrane containing lipids inside. For example, the lipid composition which forms the liposome can contain a first lipid (FFT-10) of Formula (I) and/or a second lipid (FFT-20) of Formula (II) as constituent ingredients thereof. These lipids are biodegradable lipids. Here, by adjusting the lipid composition of the lipid nanoparticles using these lipids, appropriate affinity can be achieved.

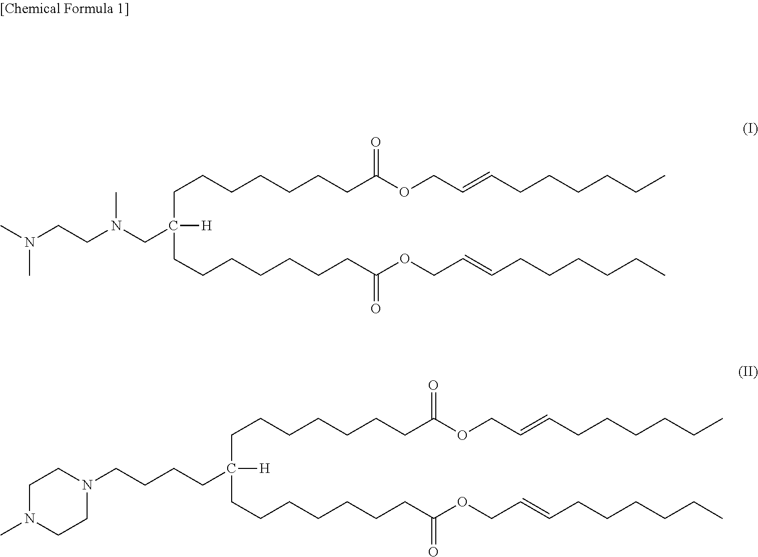

The lipid nanoparticles may also contain further lipids in addition to the first lipid and second lipid described above. Of the compositions of the lipid molecular materials that compose the lipid nanoparticles, the fraction composed by the first lipid and the second lipid is referred to as a “first fraction” hereinafter. The fraction composed by the lipid molecular materials other than the first lipid and the second lipid is referred to as a “second fraction” hereinafter. The lipids contained in the second fraction are collectively referred to as a “third lipid” as well hereinafter.

The terms “first fraction” and “second fraction” refer to the composition of the constituent ingredients of the lipid nanoparticles, and do not indicate the physical location of the lipids contained therein. For example, the constituent ingredients of the first fraction and the second fraction each do not need to be in a single group within the lipid nanoparticles, and the lipids contained in the first fraction and the lipids contained in the second fraction can exist in a mixed state. The composition ratio of the first fraction to the total lipid material that constitutes the lipid nanoparticle can be 5% or more, 10% or more, 15% or more, for example, 10% to 80% or 15% to 60%.

In other words, the total content of FFT-10 and/or FFT-20 can be 5% or more, 8% or more, 10% or more, 15% or more, for example, 10% to 60%, or 15% to 60%, or 15% to 50%, as a composition ratio of lipid nanoparticles. The maximum content of FFT-10 and FFT-20 in the lipid nanoparticles can be, for example, such an amount that the lipid nanoparticles can form liposomes. The composition ratio of the second lipid in the first fraction can be 0% to 100%, for example, 15% to 75%, 20% to 60%, 24% to 50%, etc. Similarly, the composition ratio of the first lipid in the first fraction can be 0% to 100%, for example, 15% to 75%, 20% to 60%, 24% to 50%, etc. Here, percentages are expressed in moles/mole percent unless otherwise stated.

The particle diameter of the lipid nanoparticles and the permeability thereof into cells may vary depending on the mixing ratio of the first lipids and the second lipids in the first fraction. For example, the particle diameter of the lipid nanoparticles can increase as the amount of the second lipids increases. The average particle diameter of the lipid nanoparticles may be changed depending on the usage. For example, it may be adjusted to about 50 nm to about 300 nm. For example, it may be about 70 nm to about 100 nm.

The type of the third lipids contained in the second fraction of the lipid nanoparticle is not limited to that mentioned above, but, for example, the second fraction contains a base lipid. As the base lipid, for example, a lipid that is a main ingredient of a biological membrane can be used. Such base lipids include phospholipids or sphingolipids, such as diacyl phosphatidyl choline, diacyl phosphatidyl ethanolamine, ceramide, sphingomyelin, dihydrosphingomyelin, cephalin, or cerebroside, or any combination of those listed.

For example, as base lipids, 1,2-dioleoyl-sn-glycero-3-phosphoethanolamine (DOPE), 1,2-stearoyl-sn-glycero-3-phosphoethanolamine (DSPE), 1,2-dipalmitoyl-sn-glycero-3-phosphatidylcholine (DPPC), 1-palmitoyl-2-oleoyl-sn-glycero-3-phosphatidylcholine (POPC), 1,2-di-O-octadecyl-3-trimethylammonium propane (DOTMA), 1,2-dioleoyl-3-dimethylammonium propane (DODAP), 1,2-Dimyristoyl-3-dimethylammonium propane (14:0 DAP), 1,2-Dipalmitoyl-3-dimethylammonium propane (16:0 DAP), 1,2-Distearoyl-3-dimethylammonium propane (18:0 DAP), N-(4-Carboxybenzyl)-N, N-dimethyl-2, 3-bis(oleoyloxy)propane (DOBAQ), 1,2-Di-oleoyl-3-trimethylammonium propane (DOTAP), 1,2-Di-oleoyl-sn-glycero-3-phosphocholine (DOPC), 1,2-Dilinoleoyl-sn-glycero-3-phosphocholine (DLPPC), 1,2-Dioleoyl-sn-glycero-3-phospho-L-serine (DOPS), or cholesterol, or a combination of any of these, should preferably be used. It is preferable to use cationic lipids or neutral lipids as the base lipid mentioned above, and the acid dissociation constant of the lipid nanoparticles can be adjusted by the content of the lipid. For example, it is also preferable to use DOTAP as a cationic lipid, and it is also preferable to use DOPE as a neutral lipid.

The composition ratio of cationic lipids such as FFT10, FFT20 and DOTAP to the whole amount of the lipid nanoparticles should preferably be, for example, 65% or less, in order to adjust the appropriate affinity to the target cell. For example, it may be in a range of 5% to 65%, 10% to 65%, 20% to 65%, 30% to 65%, or 40% to 65%. Further, the adjustment of the lipid composition to obtain the appropriate affinity, can be achieved by changing the constituent ingredient ratio of the cationic lipids to be contained in the lipid nanoparticles or creating a gradient therein, depending on, for example, the type and state of the target cell. In order to achieve an appropriate affinity for target cells in a specific state, it is sufficient to adjust the constituent ingredient ratio of the cationic lipid, and it is also possible to achieve the appropriate affinity for target cells in each state by changing the constituent ingredient ratio of the cationic lipid in the first lipid nanoparticle and the second lipid nanoparticle. For example, it is possible to achieve an appropriate affinity by making the constituent ingredient ratio of cationic lipids contained in the second lipid nanoparticle greater than the constituent ingredient ratio of cationic lipids in the lipid nanoparticle of the first lipid nanoparticle.

It is also preferable that the second fraction should contain lipids that prevent aggregation of the lipid nanoparticles. For example, the lipids that prevent aggregation may further include PEG-modified lipids, such as polyethylene glycol (PEG), dimyristoylglycerol (DMG-PEG), polyamide oligomers derived from omega-amino (oligoehtylene glycol) alkanoic acid monomers (U.S. Pat. No. 6,320,017 B), monosialogangliosides, or the like.

The second fraction may further contain lipids having relatively low toxicity for adjusting toxicity; lipids having functional groups that bind ligands to lipid nanoparticles; and lipids for inhibiting the leakage of internal contents such as sterols, for example, cholesterol. In particular, it is preferable to contain cholesterol.

The type and composition of the lipids used in the second fraction may be selected appropriately, in consideration of the acid dissociation constant (pKa) of the lipid nanoparticles to be produced, the particle diameter of the lipid nanoparticles, the type of activator to be included, or the stability of the particles in cells, etc.

For example, the second fraction is preferable because it has particularly high efficiency in delivering the activator, when it contains DOPE, DOTAP, cholesterol, and DMG-PEG.

In addition to the activator, the lipid nanoparticles may encapsulate further components as needed. Further components may include, for example, pH regulators, osmotic pressure regulators, and gene activators. The pH regulators include, for example, organic acids such as citric acid and the like and salts thereof. The osmotic pressure regulators include sugars or amino acids. Here, the gene activators can be substances that promote or support the activity of the activator when the activator is a gene.

Lipid nanoparticles that encapsulate the activator and other substances as needed can be produced using conventionally known methods used, for example, when encapsulating small molecules in lipid nanoparticles, such as the Bangham method, organic solvent extraction method, surfactant removal method, or freeze-thaw method. For example, a lipid mixture obtained by adding a desired ratio of a lipid nanoparticle material to an organic solvent such as alcohol, and an aqueous buffer solution containing an ingredient to be encapsulated, such as an activator, are prepared, and the aqueous buffer solution is added to the lipid mixture. Here, by agitating the thus obtained mixture for suspension, lipid nanoparticles that encapsulate the activator and the like are formed.

With use of such lipid nanoparticle, it is possible to improve the rate of delivery an activator gene to cells. Further, the rate of delivery of an activator to cells can be made more uniform, thus making it easier to control the quality.

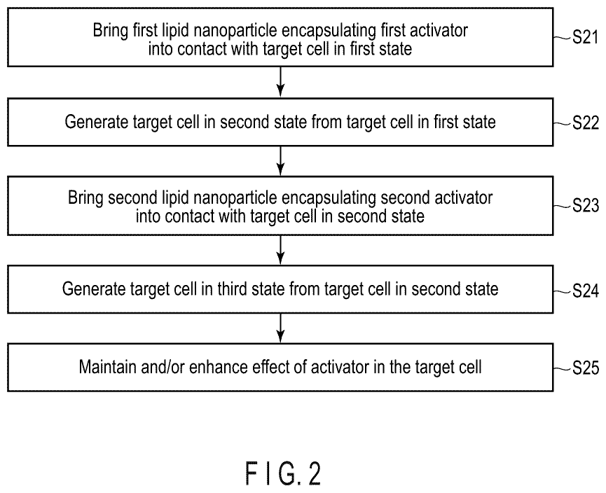

The method described herein is a method for activating target cells using the delivery carrier set 1001 described above. The method may be a method for maintaining and/or enhancing the effect of the activator in the target cell. Specifically, as shown in FIG. 2, the method includes at least the following steps. That is, the first lipid nanoparticle encapsulating the first activator is brought into contact with the target cell in the first state (S21), the target cell in the second state is generated from the target cell in the first state (S22), the second lipid nanoparticle encapsulating the second activator is brought into contact with the target cell in the second state (S23), the target cell in the third state is generated from the target cell in the second state (S24), and the effect of the activator in the target cell is maintained and/or enhanced (S25). Here, the first lipid nanoparticle has a lipid composition designed to exhibit an appropriate affinity for the target cells in the first state. The lipid composition of the second lipid nanoparticle is different from the lipid composition of the first lipid nanoparticle and has a lipid composition designed to exhibit an appropriate affinity for the target cells in the second state. The second activator may be of the same type as the first activator or a different type therefrom.

The steps S21 and S23 can be carried out, for example, by adding the activator to the aqueous solution when containing the target cells. The steps S22 and S24 can be carried out by bringing into contact with the activator of the steps S21 and S23 and/or by letting it stand after the contact with the cells, that is, for example, by incubation. The change of the state of the target cell from its initial state to a different state, that is, from the first state to the second state, and from the second state to the third state, includes such changes that are observable or not observable, in the appearance of the target cell, for example, its overall and partial morphology, its properties such as color and hue, and the presence amount, distribution, kinetics, functional changes, changes in terms of function, and the presence or absence of synthesis of specific intracellular substances, magnitude of synthesis ability, amount of synthesis of, for example, amino acids, peptides, proteins, RNA, salts, functional components, and non-functional components, etc.

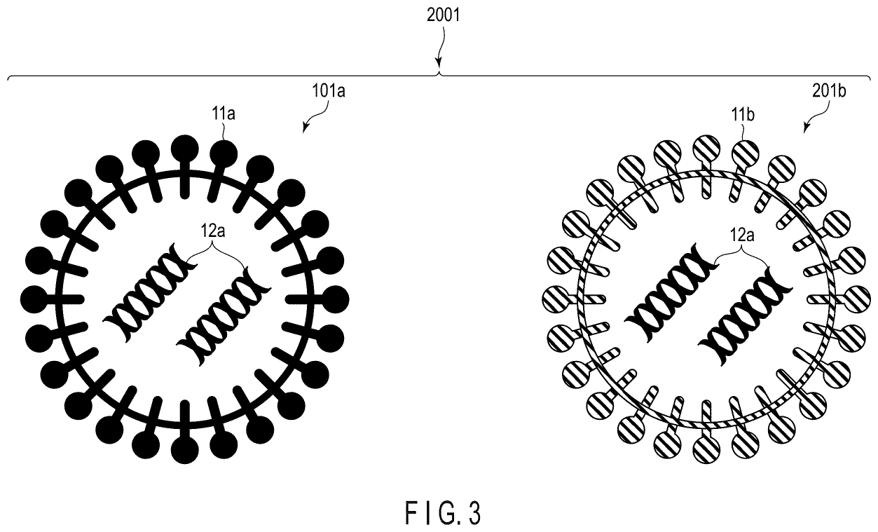

For example, when the effect of an activator causes a change in the target cell that is not observable, the method can maintain or enhance such an effect. For such a purpose, for example, a delivery carrier set 2001 according to a further embodiment as shown in FIG. 3 may be used. FIG. 3 is a cross-sectional diagram showing a further example of the delivery carrier set. The delivery carrier set 2001 comprises a first delivery carrier 101a and a second delivery carrier 201b. In this case, the first delivery carrier 101a is one as described above. In this case, the second delivery carrier 201b comprises lipid nanoparticles 11b and activators 12a. The lipid nanoparticles 11b make a hollow body formed from a lipid membrane, for example, and they are liposomes. The activators 12a are encapsulated within the lipid nanoparticles 11b, that is, within the hollow made thereby. The activators 12a are of the same type as the activators 12a encapsulated in the lipid nanoparticles 11a and are the type that exhibits the same effect. The lipid composition of the lipid nanoparticles 11b is designed to exhibit an appropriate affinity for target cells that has not been changed to an extent sufficient to be observed by the first delivery carrier 101a.

According to the method for maintaining and/or enhancing the effect of an activator in a target cell using the delivery carrier set 2001, first, a delivery carrier 101a is brought into contact with a target cell in a first state (S21), and a target cell in a second state is generated from the target cell in the first state (S22). Next, a delivery carrier 201b is brought into contact with the target cell in the second state (S23), and a target cell in a third state is generated from the target cell in the second state (S24). In this manner, for example, the expression of changes that are not observable by the activator is maintained or enhanced in the target cell. As an easily graspable example, according to this method, the activator 12a is delivered into the target cell more effectively, and therefore the effect of the activator 12a is maintained or enhanced more effectively. As a result, for example, changes that are not observable can be now observed.

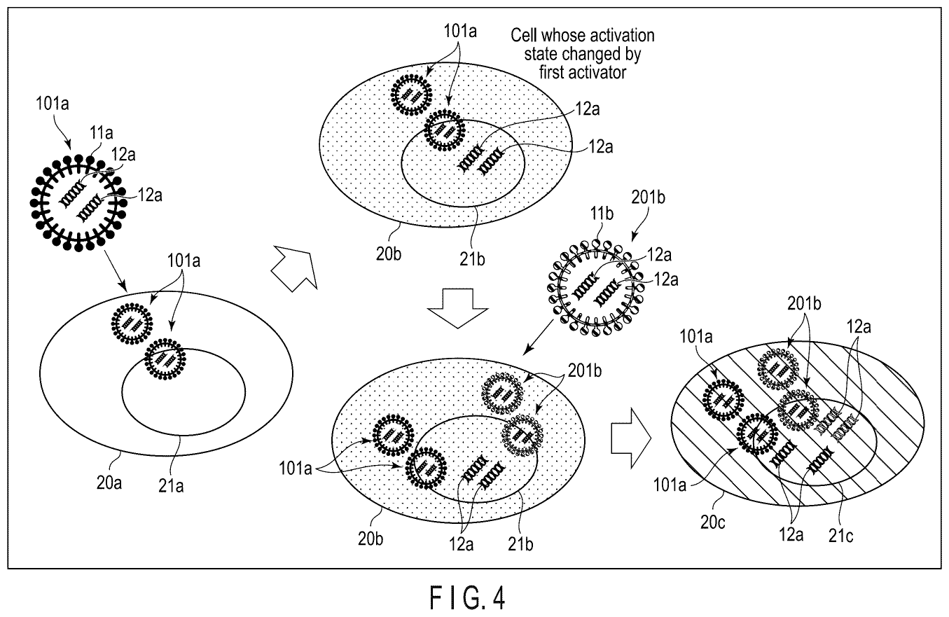

The method of the first embodiment will now be explained with reference to FIG. 4. In this example, the delivery carrier set 2001 shown in FIG. 3 is used. The target cell 20a is in the first state represented by a white outline in FIG. 4. First, when the first delivery carrier 101a is delivered into the target cell 20a, for example, the delivery carrier 101a comes into contact with the target cell 20a, enters the inside thereof, and degrades. Then, the activator 12a further proceeds towards the nucleus 21a. As a result, the target cell 20a shifts from the first state, which is represented by the white outline, to the second state, which is indicated by dots, due to the activator 12a. Next, a second delivery carrier 201b having an affinity appropriate for the second state is delivered into the target cell 20b. The delivery carrier 201b, for example, progresses into the interior of the target cell 20b in the second state, and degrades. Then, the activator 12a further progresses into its nucleus 21b. In this way, the target cell 20c shifts from the second state, represented by dots, to the third state, indicated by diagonal lines. Thus, it is possible to enhance or sustain the effect of the activators 12a of the same type in the target cell, for example, by using delivery carriers having lipid compositions that have an affinity that is suitable for the state of each respective cell. Note here that even when using delivery carriers that contain different types of activators 12a and 12b, respectively, it is also possible to sustain and/or enhance the effects of the activators 12a and/or 12b.

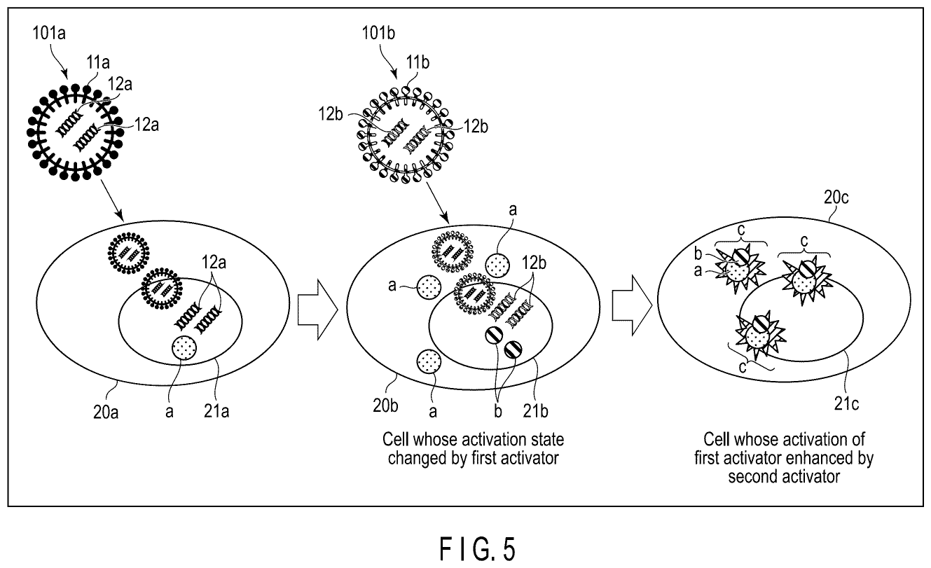

A still another example of the method will now be explained with reference to FIG. 5. In this example, the delivery carrier set 1001 shown in FIG. 1 is used. First, when the first delivery carrier 101a is delivered into the target cell 20a, the delivery carrier 101a comes into contact with the target cell 20a, enters the inside thereof, and degrades. Then, the activators 12a further proceed towards the nucleus 21a. As a result, for example, a substance a, for example, a peptide a is formed by the activators 12a. That is, due to the action of the activators 12a, the target cell 20a, which has a state that does not produce the peptide a, as the first state shifts to a target cell 20b that has a state that produces the peptide a, as the second state. Next, the second delivery carrier 101b having an appropriate affinity for this second state is delivered into the target cell 20b. The delivery carrier 101b progresses into the interior of the target cell 20b in the second state, and degrades. Then, the activators 12b further progress into the nucleus 21b. As a result, for example, a substance b, for example, a peptide b, is produced. That is, the target cell 20b, which has the second state that produces the peptide a but does not produce the peptide b, shifts to a target cell 20c having the third state that produces both the peptide a and the peptide b. According to this method, in the target cell 20c in the third state, the activity of the first activators 12a are enhanced by the second activators 12b. Such enhancement may occur, for example, due to an event occurring between the substance a and the substance b, such as an interaction between them or an action from one to the other, that is conventionally known itself. Such an action may be, for example, the binding of the peptide a and peptide b together.

According to such an embodiment, it is possible to maintain and/or enhance the desired effect of an activator in the desired target cell. For example, even when it is difficult to maintain the effect using ordinary general methods or conventional methods, or when it is difficult to observe or detect the effect, or when it is difficult to obtain the desired effect, it is possible to maintain the effect of the activator for a desired period of time, to make the effect observable or detectable, or to obtain a sufficient effect.

### Second Embodiment

The second embodiment is, simply put, a method that repeats the steps of the method of the first embodiment. More specifically, it can be said that the method is similar to the method of the first embodiment, except that it includes contact between a further activator and the target cell and further generation of target cells in a further state in addition to the method of the first embodiment. As a result of the delivery of such an activator, the state of the target cells changes sequentially. According to this method, delivery carriers having lipid nanoparticles appropriate for that state are sequentially used, and the activators contained therein are sequentially made to act.

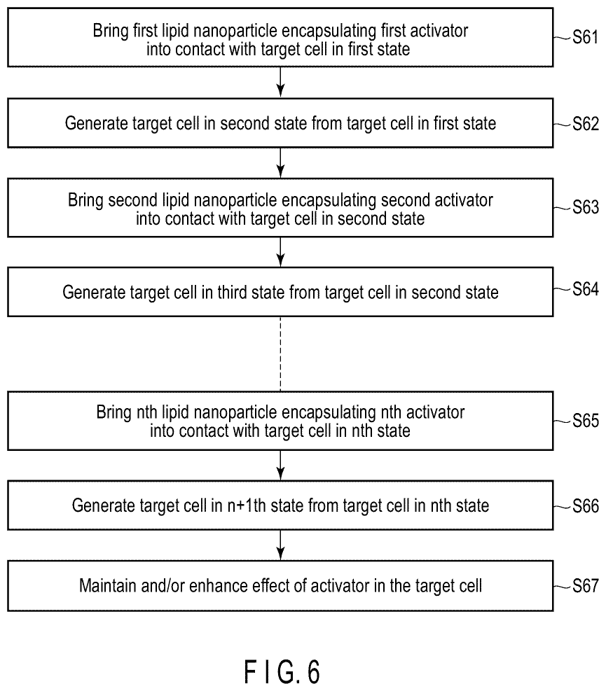

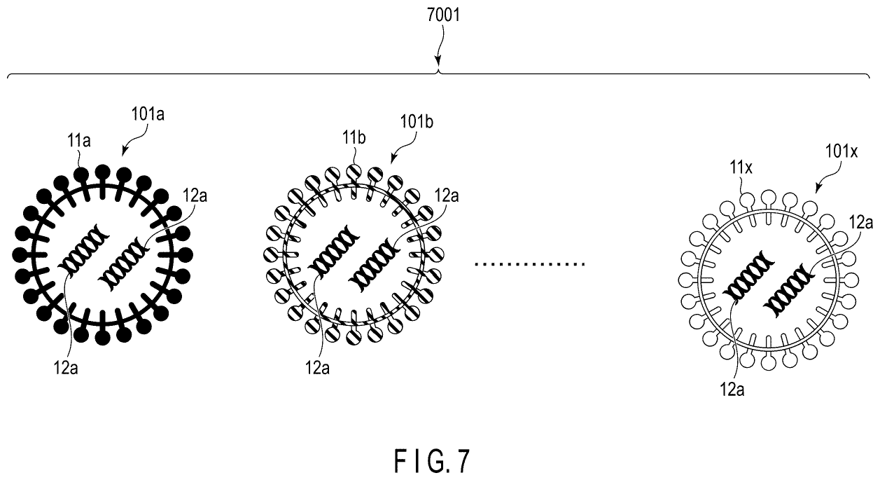

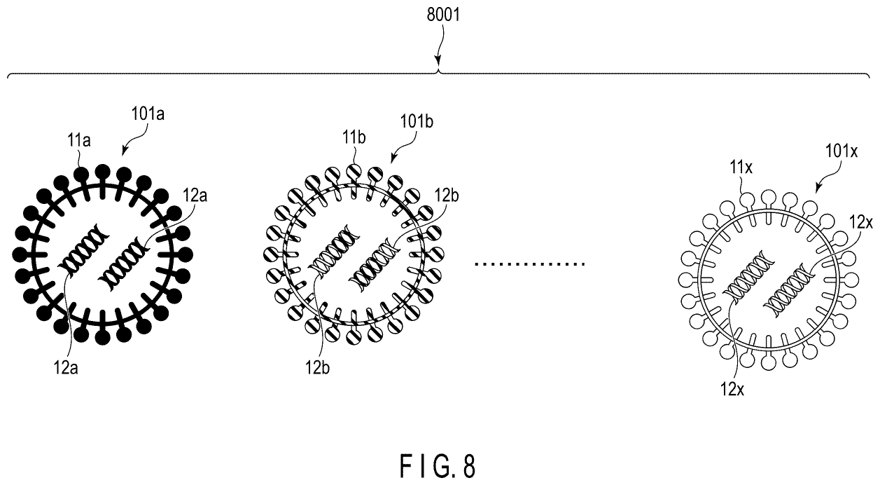

The method will be explained with reference to FIG. 6. Two examples of delivery carrier sets used in the method are shown in FIGS. 7 and 8, respectively. In the delivery carrier set 7001 shown in FIG. 7, activators 12a of the same type are encapsulated in lipid nanoparticles 11a to 11x, which are designed to have appropriate affinity for target cells in the respective states. Here, x is equal to n, which will be described later, and n is an integer greater than or equal to 3. In the delivery carrier set 8001 shown in FIG. 8, activators 12a to 12x of different types are encapsulated in the lipid nanoparticles 11a to 11x each designed to have an appropriate affinity for the respective target cells in the respective state.

In this method, the target cells are activated, for example, using the delivery carrier set 7001 or 8001. The method can be a method for maintaining and/or enhancing the effect of the activators in the target cells, for example. Specifically, as shown in FIG. 6, the method includes at least bringing the first lipid nanoparticle encapsulating the first activator into contact with the target cell in the first state (S61), generating the target cell in the second state from the target cell in the first state (S62), bringing the second lipid nanoparticle encapsulating the second activator into contact with the target cell in the second state (S63), generating the target cell in the third state from the target cell in the second state (S64), bringing the nth lipid nanoparticle encapsulating the nth activator into contact with the target cell in the nth state (S65), generating target cells in the nth+1 state from target cells in the nth state, and maintaining and/or enhancing the effect of the activator in the target cells (S67).

Here, n is an integer greater than or equal to 3, and the generation is are carried out n times sequentially from the generation of the target cells in the second state to the generation of the target cells in the n+1 state. Here, the lipid nanoparticles 1 to n each have a lipid composition designed to exhibits an appropriate affinity for the respective target cells in the corresponding one of the first to nth states. For example, all of the lipid nanoparticles 1 to n may have different lipid compositions, some of the lipid nanoparticles 1 to n may have the same composition, and some of the lipid nanoparticles may have different compositions. Further, the first to nth activators may be the same type, some of the activators may be the same type, or some of the activators may be different types. Alternatively, the first to nth activators may all be different types from each other.

Here, n is an integer greater than or equal to 3, but for example, n may be an integer from 3 to 30, an integer from 3 to 10, or an integer from 3 to 5. As described above, x is equal to n. The number of n and x, the number of states of the target cells, and the type and state of the object cells and the like may be selected according to the objectives of the practitioner.

According to the embodiment having such contents as above, it is possible to maintain and/or enhance the desired effect of an activator in the desired target cell. For example, even when it is difficult to maintain the effect using ordinary general methods or conventional methods, or when it is difficult to observe or detect the effect, or when it is difficult to obtain the desired effect, it is possible to maintain the effect of the activator for a desired period of time, to make the effect observable or detectable, or to obtain a sufficient effect. With this method, the cells of the object can be obtained.

### Third Embodiment

The third embodiment is a method for producing predetermined object cells from target cells, by utilizing the first and second embodiments described above. In the method according to the first embodiment, the target cells in the third state may be used as the object cells, and in the method according to the second embodiment, the target cells in the n+1 state may be used as the object cells. In other words, the method should only be designed in advance so that the object cells become target cells in the third state or target cells in the n+1 state.

### Fourth Embodiment

The fourth to seventh embodiments described below are delivery carrier sets for use in any of the above-described methods. For example, the delivery carrier set for use in the first embodiment can be a delivery carrier set such as that shown in FIG. 1 or FIG. 3. The fourth embodiment described next is a delivery carrier set shown in FIG. 1.

The delivery carrier set according to the fourth embodiment will be described with reference to FIG. 1. The delivery carrier set 1001 contains a first delivery carrier 101a and a second delivery carrier 101b. The delivery carrier 101a contains lipid nanoparticles 11a (for example, liposomes 11a) and activators 12a encapsulated therein. The lipid nanoparticles 11a have an affinity appropriate for the target cell in the first state. The delivery carrier 101b contains lipid nanoparticles 11b (for example, liposomes 11b) and activators 12b contained therein. The lipid nanoparticles 11b have an affinity appropriate for the target cell in the second state. The activators 12a and the activators 12b are different in type from each other. Here, “different types” means that they are substances of different types, but they may be substances that have the same activity or effect, or they may be substances of different types and different activities or effects, or they may be substances that are categorized in the same category, or they may be substances that are categorized in different categories. The selection of the first and second activators, or the selection of a combination thereof, may be carried out according to the target cells and/or object cells, or the desired purpose. By using the delivery carrier set 1001, the activator which should be delivered can be appropriately delivered into the target cells according to the state of the target cells. With this configuration, it is possible to maintain and/or enhance the effect of the activator which should be delivered in the target cells. In this manner, the cells of the object can be obtained more efficiently.

### Fifth Embodiment

The fifth embodiment will be explained with reference to FIG. 3. The delivery carrier set 2001 shown in FIG. 3 contains a first delivery carrier 101a and second delivery carriers 201b. The delivery carrier 101a includes lipid nanoparticles 11a (for example, liposomes 11a) and activators 12a encapsulated therein. The lipid nanoparticles 11a have an affinity appropriate for target cells in the first state. The delivery carrier 201b includes lipid nanoparticles 11b (for example, liposomes 11b) and activators 12a encapsulated therein. The lipid nanoparticles 11b have an affinity appropriate for target cells in the second state. The delivery carrier set 2001 can maintain and/or enhance the effect of the activator by repeatedly introducing activators of the same type of. Further, it is also possible thereby to efficiently obtain the desired object cells or homogeneous object cells.

### Sixth Embodiment

The sixth embodiment is used for the method according to the second embodiment, for example, as shown in FIG. 6. The sixth embodiment will be explained with reference to FIG. 7. A delivery carrier set 7001 includes first to xth delivery carriers 101a to 101x. The first delivery carrier 101a includes lipid nanoparticles 11a (for example, liposome 11a) and activators 12a encapsulated therein. The lipid nanoparticles 11a have an affinity appropriate for target cells in the first state. The delivery carrier 101b includes lipid nanoparticles 11b (for example, liposomes 11b) and the activators 12a encapsulated therein. The lipid nanoparticles 11b have an affinity appropriate for the target cell in the second state. The letter “x” corresponds to “n” of the corresponding method and is an integer greater than or equal to 3. The first to xth delivery carriers 101a to 101x correspond to the first to nth state target cells, respectively, and each have an affinity appropriate for the target cells of the respective state. Here, all of the first to xth delivery carriers contain activators 12a of the same type. With this configuration, it is easier for the activators and/or the effect of the activators to reach any of the target cells in each respective state. As the activators and the effect thereof reach the cells in layers, it is possible to maintain and/or enhance the effect of the activators 12a in the target cells. Further, it is also possible to produce object cells in a good, appropriate, efficient, or homogeneous manner.

### Seventh Embodiment

The seventh embodiment is used for a method according to the second embodiment shown in FIG. 6, for example. The seventh embodiment will be explained with reference to FIG. 8. A delivery carrier set 8001 contains the first to the xth delivery carriers 101a to 101x. The first delivery carrier 101a includes lipid nanoparticles 11a (for example, liposomes 11a) and activators 12a encapsulated therein. The lipid nanoparticles 11a have an affinity appropriate for target cells in the first state. The delivery carrier 101b includes lipid nanoparticles 11b (for example, liposomes 11b) and activators 12a encapsulated therein. The lipid nanoparticles 11b have an affinity appropriate for target cells in the second state. The letter “x” corresponds to “n” of the corresponding method and is an integer greater than or equal to 3. The first to nth lipid nanoparticles 11a to 11x correspond to the target cells of the first to nth states, respectively, and each have an affinity appropriate for the target cells of the respective state. The first to xth delivery carriers may all contain activators 12a to 12x of different types from each other, or some of the first to xth delivery carriers may contain activators of the same type. With this configuration, it is easier for the activators and/or the effect of the activators to reach any of the target cells in the respective state. Further, by sequentially delivering the appropriate activators according to the conditions of the target cells and according to the desired plan, it is possible to maintain and/or enhance the effect of the activators 12a to 12x in the target cells. Furthermore, it is also possible to produce the object cells in a good, appropriate, efficient, or homogeneous manner.

### Eighth Embodiment

The delivery carrier sets according to the fourth to seventh embodiments described above may be provided in a condition directly ready for use on the desired target cells, or it may be provided as a delivery carrier preparation kit in a form of materials that can be adjusted by the user of the above-described delivery carrier set at the time of use. In this case, the kit comprises, for example, a first activator to be delivered into target cells in the first state, a first lipid nanoparticle material for encapsulating the first activator, a second activator to be delivered into target cells in the second state, and a second lipid nanoparticle material for encapsulating the second activator. The first lipid nanoparticle has a lipid composition designed to exhibit appropriate affinity for target cells in the first state. The second activator is an activator of the same type as or different type from the first activator. The second lipid nanoparticle has a lipid composition different from that of the first lipid nanoparticle and designed to exhibit appropriate affinity for target cells in the second state.

The materials for these first and second lipid nanoparticles and the first and second activators may be provided in containers in appropriate conditions so that each is stably provided as a substance. In addition, instructions for preparing the delivery carrier for the user to properly manufacture may be further included.

There have been reports on methods for introducing multiple genes into material cells in order to create target cells. However, in practice, when introducing multiple desired genes into material cells, it is difficult to introduce all genes sufficiently and achieve sufficient activity. For example, they result commonly in that the activity of one gene becomes lower than that of the other. However, such drawbacks can be solved using any of the above-described embodiments. Further, according to any of the above-described embodiments, it is also possible to produce object cells to which activators are uniformly delivered, and it is also possible to produce object cells efficiently and/or uniformly.

### EXAMPLES

Examples of the preparation and use of the lipid nanoparticles of the embodiments will now be provided.

### Experiment 1: Preparation of Lipid Nanoparticles Encapsulating GFP Gene

As the nucleic acid to be encapsulated in the lipid nanoparticles, messenger RNA (mRNA) of green fluorescent protein (GFP) gene (manufactured by OZ Bioscience) was used. The nucleic acid was suspended in 10 mM HEPES (pH 7.3) to obtain a nucleic acid solution.

FFT-10, FFT-20, DOPE, DOTAP, cholesterol, and DMG-PEG were dissolved in ethanol each at the molar ratios of 0:25.8:4.9:9.8:55.8:3.7, respectively, to obtain a lipid solution. The lipid solution and the above-described nucleic acid solution were mixed using a microflow chip and syringe pump. After diluting the mixed solution 10-fold with 10 mM HEPES (pH 7.3), the resultant was concentrated using an ultrafiltration filter (Amicon Ultra 0.5 Ultra-Cel-50, Merck), and the lipid nanoparticles of Examples 1 to 4 were obtained.

### Experiment 2: Quantification of Lipids in Lipid Nanoparticles by LC/MS

In order to compare the composition ratio of lipids used for the preparation of the lipid nanoparticles and the composition ratio of the lipids contained in the lipid nanoparticles prepared, and to evaluate the error range of the lipid composition ratio, the quantification of lipid was carried out by liquid chromatography mass spectrometry (LC/MS). The lipid nanoparticles prepared in Experiment 1 were diluted with methanol, and using an LC/MS device (ACQUITY UPLC/QTOF System, Waters), FFT-10, FFT-20, DOPE, DOTAP, cholesterol, and DMG-PEG contained in each of the lipid nanoparticles were quantified under the conditions shown in TABLE 1.

TABLE 1

| UPLC | Transfer | 10 mM ammonium acetate | |
|---|---|---|---|
| analysis | phase A | aqueous solution | |
| conditions | Transfer | Methanol | |
| | phase B | | |
| MS | Measurement | Scan positive ion | |
| conditions | mode | | |
| | Ionization | ESI | FFT-10, FFT-20, |
| | mode | | DOPE, DOTAP, DMG-PEG |
| | | APCI | Cholesterol |

The measurements were carried out in accordance with the manual supplied with the LC/MS device. The ratio of each of the lipids was converted to molar percentage (mol %) from the lipid quantification results, and the lipid composition ratios of the lipid nanoparticles were calculated. TABLES 2 to 4 show the composition ratio of lipids prepared for the lipid nanoparticles, the lipid composition ratio calculated from the results of the LC/MS analysis, and the amount of error.

TABLE 2
Composition ratio of lipid particle prepared
for lipid nanoparticles (mol %)

| FFT-10 | FFT-20 | DOPE | DOTAP | Chol | PEG |
|---|---|---|---|---|---|
| 0.0 | 25.8 | 4.9 | 9.8 | 55.8 | 3.7 |

TABLE 3
Lipid composition ratio calculated from
results of LC/MS analysis (mol %)

| | FFT-10 | FFT-20 | DOPE | DOTAP | Chol | PEG |
|---|---|---|---|---|---|---|
| Example 1 | 0.0 | 23.3 | 7.8 | 8.8 | 56.5 | 3.7 |
| Example 2 | 0.0 | 21.8 | 8.5 | 8.6 | 57.4 | 3.7 |
| Example 3 | 0.0 | 23.9 | 7.8 | 9.6 | 55.0 | 3.7 |
| Example 4 | 0.0 | 24.7 | 7.3 | 9.8 | 54.5 | 3.8 |

TABLE 4
Error amount (mol %)

| | | | | | |
|---|---|---|---|---|---|
| FFT-10 | FFT-20 | DOPE | DOTAP | Chol | PEG |

| | | | | | |
|---|---|---|---|---|---|
| Example 1 | −2.5 | 2.9 | −1.0 | 0.7 | 0.0 |
| Example 2 | −4.0 | 3.6 | −1.2 | 1.6 | 0.0 |
| Example 3 | −2.0 | 2.9 | −0.2 | −0.8 | 0.0 |
| Example 4 | −1.2 | 2.4 | 0.0 | −1.4 | 0.1 |

The error amounts were calculated using the composition ratio of the lipid prepared as the theoretical value and the lipid composition ratio by LC/MS analysis as the actual measured value. The results indicated that the error range for the above-described lipid nanoparticle preparation method was 0.1 to 4 mol %.

### Experiment 3: Preparation of Lipid Nanoparticles Encapsulating GFP Gen

The messenger RNA of GFP gene was used as the nucleic acid to be encapsulated in the lipid nanoparticles. The nucleic acid was suspended in 10 mM HEPES (pH 7.3) to obtain a nucleic acid solution.

FFT-10, FFT-20, DOPE, DOTAP, cholesterol, and DMG-PEG were dissolved in ethanol each at the composition ratios of prepared lipids indicated in TABLE 5 to obtain a lipid solution. The lipid solution and the above-described nucleic acid solution were mixed using a microflow chip and a syringe pump. After diluting the mixed solution 10-fold with 10 mM HEPES (pH 7.3), it was concentrated using an ultrafiltration filter (Amicon Ultra 0.5 Ultracel-50, Merck), and the lipid nanoparticles of Examples 5 to 9 were obtained, respectively.

TABLE 5

| | Composition ratio of lipid particle prepared (mol %) | | | | | |
|---|---|---|---|---|---|---|
| | FFT10 | FFT20 | DOPE | DOTAP | Chol | PEG-DMG |
| Example 5 | 20 | 20 | 0 | 10 | 48 | 2 |
| Example 6 | 20 | 20 | 10 | 10 | 36 | 4 |
| Example 7 | 58 | 0 | 0 | 17 | 23 | 2 |
| Example 8 | 0 | 32 | 5 | 9 | 51 | 3 |
| Example 9 | 2 | 30 | 2 | 17 | 45 | 4 |

### Experiment 4: Measurement of Intensity of Gene Expression

Human peripheral blood mononuclear cells (PBMC, Precision For Medicine) cultured in StemSpan SFEMII medium (Stemcell technologies) containing six types of cytokines (IL-6, SCF, TPO, Flt-3L, IL-3, and G-CSF; all by Wako) were centrifuged and collected. Then, the resultants were seeded on a 96-well culture plate to prepare a medium of 100 μL/well at 4×10⁴ cells/well of Stem Fit AK03N (with the addition of Rock Inhibitor Y27632), and each of the lipid nanoparticles prepared in Experiment 3 was added to the respective well to make 1.3 μL/well. In each case, the culture plate was placed in an incubator and the cells were cultured at 37° C. in an atmosphere of 5% CO².

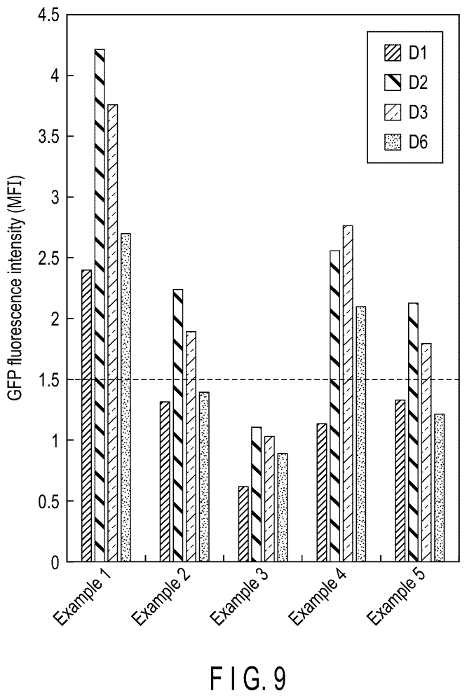

From the day after the addition of the lipid nanoparticles to the sixth day, the fluorescence intensity of GFP protein expressed from the GFP gene was photographed using a fluorescence microscope (KEYENCE BZ-X810), and the average brightness in each well was measured using image processing software (ImageJ). The measurements were taken according to the manual supplied with the fluorescence microscope. FIG. 9 shows the results of the measurement of the relative fluorescence intensity of the GFP protein.

Of the five types of lipid nanoparticles (Examples 5 to 9) having different lipid composition ratios of prepared lipids, only the lipid nanoparticles (Example 5) with the composition ratio of FFT-10:FFT-20 DOPE:DOTAP:cholesterol:DMG-PEG=20:20:0:10:48:2 (mol %) showed a relative fluorescence intensity of approximately 2.5 on the first day of measurement (D1), exceeding the reference value of 1.5, and maintained a relative fluorescence intensity of 2.5 or higher until the sixth day of measurement (D6). None of the other lipid nanoparticles (Examples 6 to 9) showed a fluorescence intensity that exceeded the measurement results of Example 5. From the results provided above, it was demonstrated that the lipid nanoparticles composed of the lipid composition ratio of Example 5 can deliver nucleic acids to PBMCs cultured under the above conditions at the highest efficiency.

### Experiment 5: Comparison of Lipid Composition with Respect to DNA Recombination Efficiency in Breast Cancer Cells

Such a system was constructed as to detect the DNA recombination performance of cells by using two genes working simultaneously, with reference to the decrease in luminescence as an indicator. Using this system, the DNA recombination efficiency in breast cancer cells was compared with use of a set of delivery carriers having different lipid compositions.

As the first lipid nanoparticle for the delivery carrier set, liposomes encapsulating the DNA recombination enzyme gene (Crea gene (NCBI Reference Sequence: NC_00585 6.1) was synthesized and inserted it into pCDNA4/V5-HisB (manufactured by Thermo Fisher Scientific)) was prepared. As the second lipid nanoparticle, liposomes encapsulating a luminescent gene (loxP sequence 5′-ATAACTCGTAATAGCACT ACATTAACGAGTTAT-3′ (sequence number 1) was synthesized to the 3′ side and 5′ side of the NLuc gene and inserted into pCDNA4/V5-HisB) were prepared. Specifically, FFT-10, FFT-20, DOPE, DOTAP, cholesterol, and DMG-PEG were dissolved in ethanol each at the composition ratios of prepared lipids shown in TABLE 6 to obtain a lipid solution. The lipid solution and the above-described nucleic acid solution were mixed using a microflow chip and a syringe pump. After diluting the mixed solution 10-fold with 10 mM HEPES (pH 7.3), it was concentrated using an ultrafiltration filter (Amicon Ultra 0.5 Ultra-Cel-50, manufactured by Merck) to obtain lipid nanoparticles that encapsulate either a DNA recombinant enzyme gene or a luminescent gene, respectively, within the liposomes of either Example 10-1, Example 10-2, or Example 10-3. Breast cancer cells were used as the target cells. The liposomes were delivered into the cells by adding the first lipid nanoparticles to the breast cancer cells cultured in a culture solution on a culture dish (MEM medium containing 10% fetal bovine serum), and then adding the second lipid nanoparticles thereto 24 hours later. After another 24 hours, the amount of luminescence was measured for the target cancer cells using a luminescence measurement device (Infinite F200 PRO plate reader (manufactured by Tecan)).

TABLE 6

| | Composition ratio of lipid particle prepared (mol %) | | | | | |
|---|---|---|---|---|---|---|
| | | | | | | PEG- |
| | FFT10 | FFT20 | DOTAP | DOPE | Chol | DMG |
| Example 10-1 | 0 | 32 | 9 | 4 | 51 | 3 |
| Example 10-2 | 32 | 0 | 6 | 9 | 51 | 3 |
| Example 10-3 | 31 | 15 | 17 | 9 | 25 | 3 |

Cationic lipid

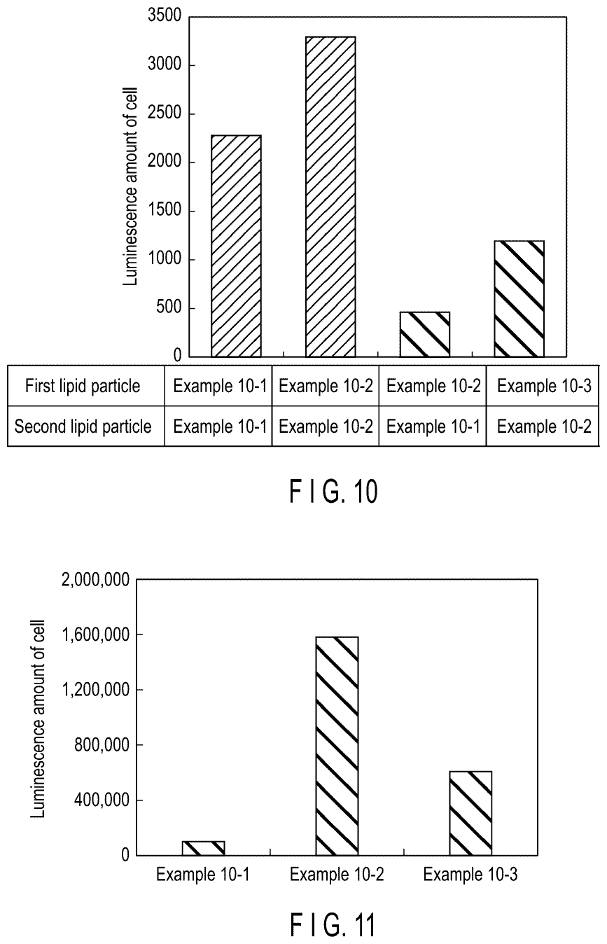

The results are shown in FIGS. 10 and 11. In the graph shown in FIG. 10, the vertical axis indicates the amount of luminescence, and the measured luminescence amount is plotted on the vertical axis. The conditions for each graph are as follows. The leftmost column in FIG. 10 shows the results obtained in the case where the first lipid nanoparticles (liposomes) encapsulating a DNA recombinant enzyme gene and having the lipid composition of Example 10-1 and the second lipid nanoparticles (liposomes) encapsulating a luminescence gene and having the lipid composition of Example 10-1 were sequentially delivered into breast cancer cells, which are the target cells. The second column from the left shows the results obtained in the case where the first lipid nanoparticles (liposomes) encapsulating a DNA recombination enzyme gene and the lipid composition of Example 10-2 and the second lipid nanoparticles (liposomes) encapsulating a luminescence gene and the lipid composition of Example 10-2 were sequentially delivered into breast cancer cells, which were the target cells. These two cases described above are comparative examples, in which the lipid compositions of the liposomes successively delivered into the target cells are the same as each other.

The third and fourth data from the left are examples. In the examples, the lipid composition of the first lipid nanoparticles delivered at first into the target cells is different from that of the second lipid nanoparticles. In each case, the composition of each is adjusted to be appropriate for the state of the target cells. More specifically, the third column from the left indicates the results of the case where the first lipid nanoparticles (liposomes) encapsulating a DNA recombination enzyme gene and having the lipid composition of Example 10-2 and the second lipid nanoparticles (liposomes) encapsulating a luminescent gene and having the lipid composition of Example 10-1 were sequentially delivered into breast cancer cells, which were the target cells. The fourth column from the left shows the results of the case where the first lipid nanoparticles (liposomes) encapsulating a DNA recombination enzyme gene and having the lipid composition of Example 10-3, and the second lipid nanoparticles (liposomes) encapsulating a luminescence gene and having the lipid composition of Example 10-2 were sequentially delivered into breast cancer cells, which were the target cells.

In the experiments conducted, when the first lipid nanoparticles (liposomes) and the second lipid nanoparticles (liposomes) progressed into the interior of the target cells and exhibited their effects, the amount of luminescence emitted by the cells decreased. When the delivery of these lipid nanoparticles is insufficient and/or the activity of the activators is low, the amount of luminescence emitted by the cells would be greater. As shown in FIG. 10, in an assay system for measuring the genomic recombination activity of breast cancer cells in which the amount of luminescence is suppressed by the cooperative action of two genes, the amount of luminescence obtained was suppressed by 6.9 times by setting the lipid compositions of the first lipid nanoparticles and the second lipid nanoparticles to be different from each other, as compared to the case where the lipid compositions of the first lipid nanoparticles and the second lipid nanoparticles were set to be the same as each other. From the graph in FIG. 10, it is clear that the activity of the activators is increased by changing the lipid composition of the first lipid nanoparticles from that of the second lipid nanoparticles or vice versa.

FIG. 11 shows the results of measuring the amount of gene delivery when lipid nanoparticles having the lipid compositions of Example 10-1, Example 10-2, and Example 10-3 were added solely to cultured breast cancer cells. The amount of gene delivery was measured using the Nano-Glow Luciferase Assay System (Promega Corporation) by reading the luminescence of each cell using a plate reader.

When comparing the lipid compositions of Example 10-1, Example 10-2 and Example 10-3, it is found that the initial transfection rates of the breast cancer cells were higher in the order of Example 10-2, Example 10-3 and Example 10-1. However, as is clear from FIG. 10, even if the lipid composition of Example 10-2, which has the highest transfection rate, were used as the lipid compositions of the first lipid nanoparticles and the second lipid nanoparticles, the activators were not delivered well. From the results obtained as above, it is clear that the effect of the activator contained in the lipid nanoparticles, that is, the liposomes, can be enhanced by sequentially introducing multiple lipid nanoparticles having different lipid compositions into the target cells.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Further examples of the embodiments will be provided as follow.

(1) A method for maintaining and/or enhancing an effect of an activator in a target cell, the method comprising, at least:
bringing a first lipid nanoparticle encapsulating a first activator into contact with the target cell in a first state;
generating a target cell in a second state from the target cell in the first state;
bringing a second lipid nanoparticle encapsulating a second activator into contact with the target cell in the second state;
generating a target cell in a third state from the target cell in the second state, and maintaining and/or enhancing the effect of the activator in the target cell,
wherein
the first lipid nanoparticle has a lipid composition designed to exhibit an affinity appropriate for the target cell in the first state, and
the second lipid nanoparticle has a lipid composition different from the lipid composition of the first lipid nanoparticle, which is designed to exhibit an affinity appropriate for the target cell in the second state, and
the second activator is of a same type as or a different type from that of the first activator.

(2) The method according to clause (1), wherein
the second state is a state in which the effect of the first activator is exhibited in the target cell in the first state, and the third state is a state in which the effect of the second activator is exhibited in the target cell in the second state.

(3) The method according to clause (1) or (2) wherein,
the first and/or second lipid nanoparticles are lipid nanoparticles composed of a lipid component containing biodegradable lipid FFT-10 and/or FFT-20.

(4) The method according to any one of clause (1) to (3) characterized in that:
a ratio of cationic lipids contained in the first and/or second lipid nanoparticles is 65% or less.

(5) The method according to any one of clause (1) to (4) characterized in that:
a component ratio of cationic lipids contained in the second lipid nanoparticle is greater than a component ratio of cationic lipids contained in the first lipid nanoparticle.

(6) The method according to any one of clause (1) to (5) characterized in that:
the first and/or second activator is a substance having an activity selected from the group consisting of protein synthesis activity, protein cleavage activity, activity affecting a state of protein expression, enzyme activity, anti-cancer activity, reprogramming activity, nucleic acid cleavage activity, gene recombination activity, and genome editing activity.

(7) The method according to any one of clause (1) to (6) characterized in that:
the method further comprises bringing nth lipid nanoparticles encapsulating an nth activator into contact with a target cell in an nth state, and generating a target cell in an (n+1)th state from the target cell in the nth state,
where n is an integer greater than or equal to 3, and n times of generation is carried out sequentially from the generation of the target cell of the second state to the generation of the target cell of the n+1 state, and
the first to nth lipid nanoparticles each have a lipid composition designed to exhibit an affinity appropriate for the respective target cell of the corresponding state.

(8) A method for producing an object cell from the target cell, the method comprising, at least:
bringing a first lipid nanoparticle encapsulating a first activator into contact with the target cell in a first state;
generating a target cell in a second state from the target cell in the first state;
bringing a second lipid nanoparticle encapsulating a second activator into contact with the target cell in the second state;
generating a target cell in a third state from the target cell in the second state,
wherein
the target cell in the third state is a predetermined target cell,
the first lipid nanoparticle has a lipid composition designed to exhibit an affinity appropriate for the target cell in the first state,
the second lipid nanoparticle has a lipid composition different from the lipid composition of the first lipid nanoparticle, which is designed to exhibit an affinity appropriate for the target cell in the second state, and
the second activator is of a same type as or a different type from that of the first activator.

(9) The method according to clause (8), wherein
the second state is a state in which the effect of the first activator is exhibited in the target cell in the first state, and the third state is a state in which the effect of the second activator is exhibited in the target cell in the second state.

(10) The method according to clause (8) or (9) wherein,
the first and/or second lipid nanoparticles are lipid nanoparticles composed of a lipid component containing biodegradable lipid FFT-10 and/or FFT-20.

(11) The method according to any one of clause (8) to (10) characterized in that:
a ratio of cationic lipids contained in the first and/or second lipid nanoparticles is 65% or less.

(12) The method according to any one of clause (8) to (11) characterized in that:
a component ratio of cationic lipids contained in the second lipid nanoparticle is greater than a component ratio of cationic lipids contained in the first lipid nanoparticle.

(13) The method according to any one of clause (8) to (12) characterized in that:
the first and/or second activator is a substance having an activity selected from the group consisting of protein synthesis activity, protein cleavage activity, activity affecting a state of protein expression, enzyme activity, anti-cancer activity, reprogramming activity, nucleic acid cleavage activity, gene recombination activity, and genome editing activity.

(14) The method according to any one of clause (8) to (13) characterized in that:
the method further comprises bringing nth lipid nanoparticles encapsulating an nth activator into contact with a target cell in an nth state, and generating a target cell in an (n+1)th state from the target cell in the nth state,
where n is an integer greater than or equal to 3, and n times of generation is carried out sequentially from the generation of the target cell of the second state to the generation of the target cell of the n+1 state, and
the first to nth lipid nanoparticles each have a lipid composition designed to exhibit an affinity appropriate for the respective target cell of the corresponding state.

(15) A delivery carrier set for use in any one of the methods according to clause (1) to (14), comprising:
a first activator to be delivered into a target cell in the first state;
a first lipid nanoparticle encapsulating the first activator and having a lipid composition designed to exhibit an affinity appropriate for the target cell in the first state;
a second activator to be delivered into a target cell in the second state, which is the same type as or a different type from that of the first activator;
a second lipid nanoparticle encapsulating the second activator and having a lipid composition different from that of the first lipid nanoparticle and designed to exhibit an affinity appropriate for the target cell in the second state.

(16) A delivery carrier set for use in the method according to clause (7) or (14), further comprising:
first to nth activators of a same type or different types, to be delivered respectively into corresponding target cells in first to nth states, respectively; and
first to nth lipid nanoparticles corresponding to the first to nth activators, respectively, each encapsulating any one of the first to nth activators,
wherein
the first to nth lipid nanoparticles each having a lipid composition designed to exhibit an affinity appropriate for the respective target cell in the corresponding state.

(17) A kit for preparing a delivery carrier for use in the method according to clause (1), comprising:
a first activator to be delivered into a target cell in the first state;
a first lipid nanoparticle material for encapsulating the first activator, which has a lipid composition designed to exhibit an affinity appropriate for the target cell in the first state;
a second activator of a same type as or a different type from that of the first activator, which should be delivered into the target cell in the second state, and
a second lipid nanoparticle material for encapsulating the second activator, which has a lipid composition different from that of the first lipid nanoparticle and is designed to exhibit an affinity appropriate for the target cell in the second state.

(18) The kit according to clause (17), wherein
the target cells are selected from the group consisting of cells isolated from humans, cells which can proliferate when isolated, isolated cells of human origin, cells with nuclei, fibroblasts, cancer cells, and breast cancer cells, and the first and second activators are each a nucleic acid substance encoding a gene having an activity selected from the group consisting of protein synthesis activity, protein cleavage activity, anti-cancer activity, reprogramming activity, nucleic acid cleavage activity, gene recombination activity, and genome editing activity.

(19) A kit for preparing a delivery carrier for use in the method according to clause (7) or (14), comprising:
first to nth activators of a same type or different types, which should be delivered respectively into corresponding target cells in the respective first to nth states; and
first to nth lipid nanoparticle materials which correspond to the first to n activators, respectively, for encapsulating any one of the first to nth activators, each having a lipid composition designed to exhibit an affinity appropriate for the corresponding target cell in the respective state,
wherein
n is an integer greater than or equal to 3.

(20) The kit according to any one of clauses (17) to (19), wherein
the target cells are selected from the group consisting of cells isolated from humans, cells which can proliferate when isolated, isolated cells of human origin, cells with nuclei, fibroblasts, cancer cells, and breast cancer cells, and the first and second activators are each a nucleic acid substance encoding a gene having an activity selected from the group consisting of protein synthesis activity, protein cleavage activity, anti-cancer activity, reprogramming activity, nucleic acid cleavage activity, gene recombination activity, and genome editing activity.

(Sequence Table)
(Sequence No. 1)
5′-ATAACTTCTGATACATCATATTACGAGTAT-3′

What is claimed is:

1. A method for maintaining and/or enhancing an effect of an activator in a target cell, the method comprising, at least:
bringing a first lipid nanoparticle encapsulating a first activator into contact with the target cell in a first state;
generating a target cell in a second state from the target cell in the first state;
bringing a second lipid nanoparticle encapsulating a second activator into contact with the target cell in the second state;
generating a target cell in a third state from the target cell in the second state, and maintaining and/or enhancing the effect of the activator in the target cell,
wherein
the first lipid nanoparticle has a lipid composition designed to exhibit an affinity appropriate for the target cell in the first state, and
the second lipid nanoparticle has a lipid composition different from the lipid composition of the first lipid nanoparticle, which is designed to exhibit an affinity appropriate for the target cell in the second state, and
the second activator is of a same type as or a different type from that of the first activator.

2. The method of claim 1, wherein
the second state is a state in which the effect of the first activator is exhibited in the target cell in the first state, and the third state is a state in which the effect of the second activator is exhibited in the target cell in the second state.

3. The method of claim 1, wherein
the first and/or second lipid nanoparticles are lipid nanoparticles composed of a lipid component containing biodegradable lipid FFT-10 and/or FFT-20.

4. The method of claim 1, wherein
a ratio of cationic lipids contained in the first and/or second lipid nanoparticles is 65% or less.

5. The method of claim 1, wherein
a component ratio of cationic lipids contained in the second lipid nanoparticle is greater than a component ratio of cationic lipids contained in the first lipid nanoparticle.

6. The method of claim 1, wherein
the first and/or second activator is a substance having an activity selected from the group consisting of protein synthesis activity, protein cleavage activity, activity affecting a state of protein expression, enzyme activity, anti-cancer activity, reprogramming activity, nucleic acid cleavage activity, gene recombination activity, and genome editing activity.

7. The method of claim 1, wherein
the method further comprises bringing nth lipid nanoparticles encapsulating an nth activator into contact with a target cell in an nth state, and generating a target cell in an n+1th state from the target cell in the nth state,
where n is an integer greater than or equal to 3, and n times of generation is carried out sequentially from the generation of the target cell of the second state to the generation of the target cell of the n+1 state, and
the first to nth lipid nanoparticles each have a lipid composition designed to exhibit an affinity appropriate for the respective target cell of the corresponding state.

8. A method for producing an object cell from the target cell, the method comprising, at least:
bringing a first lipid nanoparticle encapsulating a first activator into contact with the target cell in a first state;
generating a target cell in a second state from the target cell in the first state;
bringing a second lipid nanoparticle encapsulating a second activator into contact with the target cell in the second state;
generating a target cell in a third state from the target cell in the second state,
wherein
the target cell in the third state is a predetermined object cell,
the first lipid nanoparticle has a lipid composition designed to exhibit an affinity appropriate for the target cell in the first state,
the second lipid nanoparticle has a lipid composition different from the lipid composition of the first lipid nanoparticle, which is designed to exhibit an affinity appropriate for the target cell in the second state, and
the second activator is of a same type as or a different type from that of the first activator.

9. The method of claim 8, wherein
the second state is a state in which the effect of the first activator is exhibited in the target cell in the first state, and the third state is a state in which the effect of the second activator is exhibited in the target cell in the second state.

10. The method of claim 8, wherein
the first and/or second lipid nanoparticles are lipid nanoparticles composed of a lipid component containing biodegradable lipid FFT-10 and/or FFT-20.

11. The method of claim 8, wherein
a ratio of cationic lipids contained in the first and/or second lipid nanoparticles is 65% or less.

12. The method of claim 8, wherein
a component ratio of cationic lipids contained in the second lipid nanoparticle is greater than a component ratio of cationic lipids contained in the first lipid nanoparticle.

13. The method of claim 8, wherein
the first and/or second activator is a substance having an activity selected from the group consisting of protein synthesis activity, protein cleavage activity, activity affecting a state of protein expression, enzyme activity, anti-cancer activity, reprogramming activity, nucleic acid cleavage activity, gene recombination activity, and genome editing activity.

14. The method of claim 8, wherein
the method further comprises bringing nth lipid nanoparticles encapsulating an nth activator into contact with a target cell in an nth state, and generating a target cell in an (n+1)th state from the target cell in the nth state,
where n is an integer greater than or equal to 3, and n times of generation is carried out sequentially from the generation of the target cell of the second state to the generation of the target cell of the n+1 state, and
the first to nth lipid nanoparticles each have a lipid composition designed to exhibit an affinity appropriate for the respective target cell of the corresponding state.

15. A delivery carrier set for use in any one of the method of claim 1, comprising:
a first activator to be delivered into a target cell in the first state;
a first lipid nanoparticle encapsulating the first activator and having a lipid composition designed to exhibit an affinity appropriate for the target cell in the first state;
a second activator to be delivered into a target cell in the second state, which is the same type as or a different type from that of the first activator;
a second lipid nanoparticle encapsulating the second activator and having a lipid composition different from that of the first lipid nanoparticle and designed to exhibit an affinity appropriate for the target cell in the second state.

16. A delivery carrier set for use in the method of claim 14, further comprising:
first to nth activators of a same type or different types, to be delivered respectively into corresponding target cells in first to nth states, respectively; and
first to nth lipid nanoparticles corresponding to the first to nth activators, respectively, each encapsulating any one of the first to nth activators,
wherein
the first to nth lipid nanoparticles each having a lipid composition designed to exhibit an affinity appropriate for the respective target cell in the corresponding state.

17. A kit for preparing a delivery carrier for use in the method of claim 1, comprising:
a first activator to be delivered into a target cell in the first state;
a first lipid nanoparticle material for encapsulating the first activator, which has a lipid composition designed to exhibit an affinity appropriate for the target cell in the first state;
a second activator of a same type as or a different type from that of the first activator, which should be delivered into the target cell in the second state, and
a second lipid nanoparticle material for encapsulating the second activator, which has a lipid composition different from that of the first lipid nanoparticle and is designed to exhibit an affinity appropriate for the target cell in the second state.

18. The kit of claim 17, wherein
the target cells are selected from the group consisting of cells isolated from humans, cells which can proliferate when isolated, isolated cells of human origin, cells with nuclei, fibroblasts, cancer cells, and breast cancer cells, and the first and second activators are each a nucleic acid substance encoding a gene having an activity selected from the group consisting of protein synthesis activity, protein cleavage activity, anti-cancer activity, reprogramming activity, nucleic acid cleavage activity, gene recombination activity, and genome editing activity.

19. A kit for preparing a delivery carrier for use in the method of claim 14, comprising:
first to nth activators of a same type or different types, which should be delivered respectively into corresponding target cells in the respective first to nth states; and
first to nth lipid nanoparticle materials which correspond to the first to n activators, respectively, for encapsulating any one of the first to nth activators, each having a lipid composition designed to exhibit an affinity appropriate for the corresponding target cell in the respective state,
wherein
n is an integer greater than or equal to 3.

20. The kit of claim 19, wherein
the target cells are selected from the group consisting of cells isolated from humans, cells which can proliferate when isolated, isolated cells of human origin, cells with nuclei, fibroblasts, cancer cells, and breast cancer cells, and the first and second activators are each a nucleic acid substance encoding a gene having an activity selected from the group consisting of protein synthesis activity, protein cleavage activity, anti-cancer activity, reprogramming activity, nucleic acid cleavage activity, gene recombination activity, and genome editing activity.


SEQUENCE LISTING (1 sequence)

SEQ ID NO: 1 - DNA, 34 bp, synthetic construct
  source 1..34: mol_type = other DNA
ataacttcgtatagcatacattatacgaagttat